(19) United States
(12) Patent Application Publication
(10) Pub. No.: US 2025/0111532 A1
(43) Pub. Date: Apr. 3, 2025

(54) IMAGE PROCESSING APPARATUS, IMAGE PROCESSING METHOD, AND PROGRAM

(71) Applicant: FUJIFILM CORPORATION, Tokyo (JP)

(72) Inventors: Hisatsugu HORIUCHI, Tokyo (JP); Takeyasu KOBAYASHI, Tokyo (JP); Tomohiro NOMURA, Tokyo (JP); Ryoji SASADA, Tokyo (JP); Koji TANINAI, Tokyo (JP)

(21) Appl. No.: 18/815,865

(22) Filed: Aug. 27, 2024

(30) Foreign Application Priority Data
Sep. 29, 2023 (JP) 2023-169563

(51) Int. Cl. G06T 7/73; A61B 6/00; A61B 6/46

(52) U.S. Cl. CPC G06T 7/73; A61B 6/465; A61B 6/488; G06T 2207/10016; G06T 2207/10121; G06T 2207/30004; G06T 2207/30196

(57) ABSTRACT

The image processing apparatus includes at least one processor. The processor acquires an optical image of an examinee; extract a plurality of feature points of a body of the examinee based on the optical image, generates a line segment determined based on any of the plurality of feature points as posture information, which is information indicating a posture of the examinee, generates a line segment corresponding to the line segment as the posture information as guide information, which is information for guiding a posture to be taken by the examinee, and displays the posture information and the guide information.

### CROSS-REFERENCE TO RELATED APPLICATIONS

The present application claims priority under 35 U.S.C. § 119 to Japanese Patent Application No. 2023-169563, filed on Sep. 29, 2023. The above application is hereby expressly incorporated by reference, in its entirety, into the present application.

### BACKGROUND

### 1. Technical Field

The disclosed technology relates to an image processing apparatus, an image processing method, and a program.

### 2. Description of the Related Art

The following techniques are known as techniques for estimating a posture of a subject from an image. For example, JP2016-178531A discloses that a posture determination unit detects a position of a pixel located at the top in a vertical direction among pixels corresponding to a person portion for each pixel column in the vertical direction of a binary image, and detects a shoulder line or a center position of a body of a person to be imaged based on a change in a case in which the detected position of the pixel is plotted in a horizontal direction.

JP2017-047105A discloses an exercise menu provision system comprising an imaging unit that images a posture of a user, a specific part extraction unit that extracts a specific part image indicating a plurality of specific parts from a user image captured by the imaging unit, a specific part analysis unit that analyzes the posture of the specific part corresponding to each specific part image based on the plurality of specific part images extracted by the specific part extraction unit, and an exercise menu provision unit that provides an exercise menu corresponding to the posture of each specific part analyzed by the specific part analysis unit.

WO2022/244298A discloses that an analysis unit analyzes a captured image acquired by a camera to detect skeleton information. In detecting skeleton information, each part (head, shoulder, hand, foot, and the like) of each person is recognized from the captured image, and a coordinate position of each part is calculated. The skeleton information includes information such as characteristic angles created by two or more line segments obtained by connecting points of the skeleton with lines.

### SUMMARY

A videofluoroscopic examination of swallowing is known as one type of examination using radiation images. A videofluoroscopic examination of swallowing is an examination to evaluate a process and a state when an examinee swallows a sample. In a videofluoroscopic examination of swallowing, a video composed of a series of radiation images captured by continuously irradiating an examinee with radiation is used.

In a case of capturing a radiation image, the examinee is positioned such that the posture of the examinee is set to a posture designated by an imaging menu or the like. Currently, it is checked whether or not the posture of the examinee is set to a posture as designated by the examinee through the radiation image. That is, the examinee is irradiated with radiation for positioning purposes.

The disclosed technology has been made in view of the above points, and an object thereof is to support positioning of an examinee performed in a case of capturing a radiation image, without performing irradiation with radiation.

According to an aspect of the disclosed technology, there is provided an image processing apparatus comprising at least one processor. The processor is configured to: acquire an optical image of an examinee; extract a plurality of feature points of a body of the examinee based on the optical image; generate a line segment determined based on any of the plurality of feature points as posture information, which is information indicating a posture of the examinee; generate a line segment corresponding to the line segment as the posture information as guide information, which is information for guiding a posture to be taken by the examinee; and display the posture information and the guide information.

The processor may be configured to display the posture information and the guide information to be superimposed on the optical image. The processor may be configured to cause the posture information and the guide information to follow a posture change of the examinee. The processor may be configured to generate, as the guide information, a line segment having a feature point closest to a support portion that supports the body of the examinee as a starting point, among feature points present at both ends of the line segment as the posture information.

The processor may be configured to set an orientation of the line segment as the guide information based on designation information for designating a posture to be taken by the examinee. The processor may be configured to acquire the designation information included in an imaging menu for a radiation image to be captured for the examinee. The processor may be configured to acquire the designation information input by an operation of an input unit or voice.

The processor may be configured to: extract an car of the examinee as a first feature point and extract an eye of the examinee as a second feature point based on the optical image; and generate a line segment connecting the first feature point and the second feature point as the posture information. The processor may be configured to: extract a midpoint of left and right shoulder joints of the examinee as a third feature point and extract a midpoint of left and right greater trochanters of the examinee as a fourth feature point based on the optical image; and generate a line segment connecting the third feature point and the fourth feature point as the posture information.

The processor may be configured to: set an orientation of the line segment as the guide information based on designation information for designating a posture to be taken by the examinee; acquire a series of radiation images continuously captured with guidance from the guide information for the examinee; and associate the designation information with the series of radiation images. The processor may be configured to: set an orientation of each of line segments as each of a plurality of pieces of the guide information based on a plurality of pieces of designation information for designating postures to be taken by the examinee, respectively; acquire a series of radiation images continuously captured with guidance from each of the plurality of pieces of guide information; and perform, for each piece of the plurality of pieces of designation information, processing of associating one piece of the plurality of pieces of designation information with some radiation images out of the series of radiation images, the some radiation images being captured with guidance from the guide information generated based on the one piece of designation information.

The processor may be configured to: specify an imaging direction of a radiation image to be captured for the examinee based on at least some of the plurality of feature points; and issue an alert in a case in which the specified imaging direction is different from a designated direction.

According to another aspect of the disclosed technology, there is provided an image processing method executed by at least one processor included in an image processing apparatus, the image processing method comprising: acquiring an optical image of an examinee; extracting a plurality of feature points of a body of the examinee based on the optical image; generating a line segment determined based on any of the plurality of feature points as posture information, which is information indicating a posture of the examinee; generating a line segment corresponding to the line segment as the posture information as guide information, which is information for guiding a posture to be taken by the examinee; and displaying the posture information and the guide information.

According to still another aspect of the disclosed technology, there is provided a program for causing at least one processor included in an image processing apparatus to execute:

acquiring an optical image of an examinee; extracting a plurality of feature points of a body of the examinee based on the optical image; generating a line segment determined based on any of the plurality of feature points as posture information, which is information indicating a posture of the examinee; generating a line segment corresponding to the line segment as the posture information as guide information, which is information for guiding a posture to be taken by the examinee; and displaying the posture information and the guide information.

According to the disclosed technology, it is possible to support positioning of an examinee performed in a case of capturing a radiation image, without performing irradiation with radiation.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a radiography system according to an embodiment of the disclosed technology;

FIG. 2 is a diagram showing an example of a configuration of a radiography apparatus according to the embodiment of the disclosed technology;

FIG. 3 is a diagram showing an example of a hardware configuration of an image processing apparatus according to the embodiment of the disclosed technology;

FIG. 4 is a functional block diagram showing an example of a functional configuration of the image processing apparatus according to the embodiment of the disclosed technology;

FIG. 5 is a diagram showing an example of posture information generation processing according to the embodiment of the disclosed technology;

FIG. 6 is a diagram showing an example of guide information generation processing according to the embodiment of the disclosed technology;

FIG. 7 is a flowchart showing an example of a flow of processing performed by a CPU according to the embodiment of the disclosed technology executing an image processing program;

FIG. 8 is a diagram showing posture information and guide information in a state in which positioning is completed;

FIG. 9 is a functional block diagram showing an example of a functional configuration of an image processing apparatus according to another embodiment of the disclosed technology;

FIG. 10 is a diagram showing an example of a structure of a data file generated by an association processing unit according to the embodiment of the disclosed technology;

FIG. 11 is a flowchart showing an example of a flow of processing performed by a CPU according to the other embodiment of the disclosed technology executing an image processing program;

FIG. 12 is a flowchart showing an example of a flow of processing performed by the CPU according to the other embodiment of the disclosed technology executing an image processing program;

FIG. 13 is a diagram showing an example of association processing according to the embodiment of the disclosed technology;

FIG. 14 is a functional block diagram showing an example of a functional configuration of an image processing apparatus according to still another embodiment of the disclosed technology; and

FIG. 15 is a flowchart showing an example of a flow of processing performed by a CPU according to the still another embodiment of the disclosed technology executing an image processing program.

### DETAILED DESCRIPTION

An example of embodiments of the disclosed technology will be described below with reference to the drawings. In addition, the same or equivalent components and parts in each drawing are given the same reference numerals, and duplicated descriptions will be omitted.

### First Embodiment

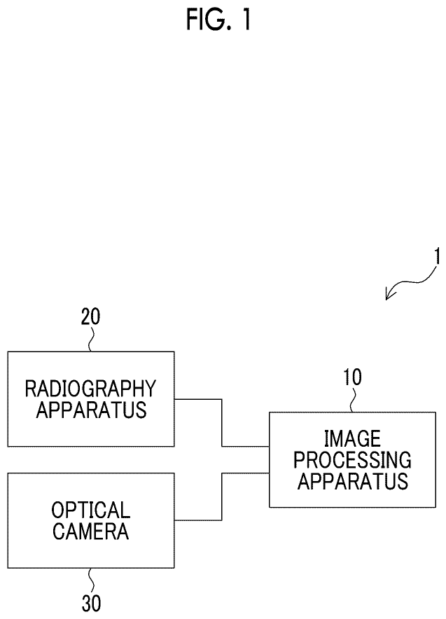

FIG. 1 is a diagram showing an example of a configuration of a radiography system 1 according to an embodiment of the disclosed technology. In the following description, an example in which the radiography system 1 is used for a videofluoroscopic examination of swallowing will be described. The radiography system 1 includes an image processing apparatus 10, a radiography apparatus 20, and an optical camera 30. The optical camera 30 may be provided integrally with the radiography apparatus 20 or may be independent from the radiography apparatus 20.

The radiography apparatus 20 is capable of imaging a radiation image using radiation such as X-rays. It is also possible to sequentially display a series of radiation images captured by performing continuous irradiation with radiation in a time series manner, thereby producing a video display. The imaging of a series of radiation images for producing a video display is called fluoroscopy. In a videofluoroscopic examination of swallowing, fluoroscopy is performed to observe the process in which an examinee swallows a sample. Note that “continuous irradiation with radiation” includes both a case in which irradiation with radiation is continuously performed over a certain period of time and a case in which irradiation with pulsed radiation is performed a plurality of times within a certain period of time. The radiation image captured by the radiography apparatus 20 is immediately transmitted to the image processing apparatus 10.

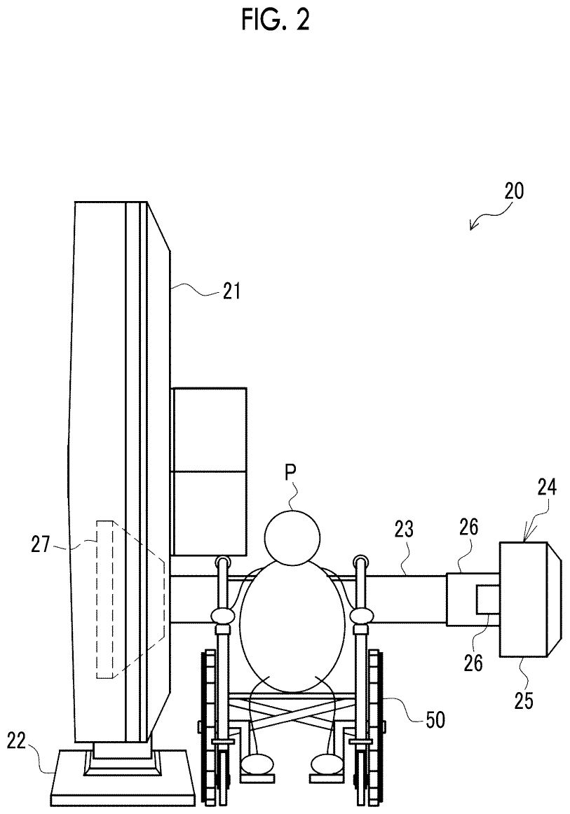

FIG. 2 is a diagram showing an example of the configuration of the radiography apparatus 20. The radiography apparatus 20 has an imaging table 21. The imaging table 21 is supported on the floor surface by a stand 22. A radiation generation unit 24 is attached to the imaging table 21 via a support column 23. The radiation generation unit 24 includes a radiation source 25 and a collimator 26. The imaging table 21 has a built-in radiation detector 27.

The radiation source 25 has a radiation tube (not shown) and irradiates an examinee P with radiation. The collimator 26 limits the irradiation field of radiation emitted from the radiation tube. The radiation detector 27 has a plurality of pixels that generate signal charges according to the radiation that has transmitted through the examinee P. The radiation detector 27 is called a flat panel detector (FPD).

The radiation generation unit 24 is capable of reciprocating together with the support column 23 along a long side direction of the imaging table 21 by a moving mechanism (not shown) such as a motor. The radiation detector 27 is capable of reciprocating along the long side direction of the imaging table 21 in conjunction with the movement of the radiation generation unit 24. The imaging table 21 and the support column 23 can be rotated between a standing state shown in FIG. 2 and a supine state (not shown) by a rotation mechanism (not shown) such as a motor. In the supine state, the surface of the imaging table 21 is parallel to the floor surface, and the direction in which the support column 23 extends is perpendicular to the floor surface. On the other hand, in the standing state, the surface of the imaging table 21 is perpendicular to the floor surface, and the direction in which the support column 23 extends is parallel to the floor surface. In the standing state, it is possible to perform fluoroscopy on the examinee P in a wheelchair 50 as shown in FIG. 2.

The optical camera 30 is an optical digital camera configured to include a complementary metal oxide semiconductor (CMOS) image sensor, a charge coupled device (CCD) image sensor, or the like, and is capable of capturing an optical image using visible light. The optical camera 30 can capture a still image and a video.

The optical camera 30 is disposed such that parts of the body necessary for estimating the posture of the examinee are within the imaging visual field. The optical camera 30 may, for example, be attached to a housing of the radiation source 25. Furthermore, the imaging direction of the optical image by the optical camera 30 and the imaging direction of the radiation image by the radiography apparatus 20 may be substantially the same. The optical camera 30 may be provided separately from the radiography apparatus 20.

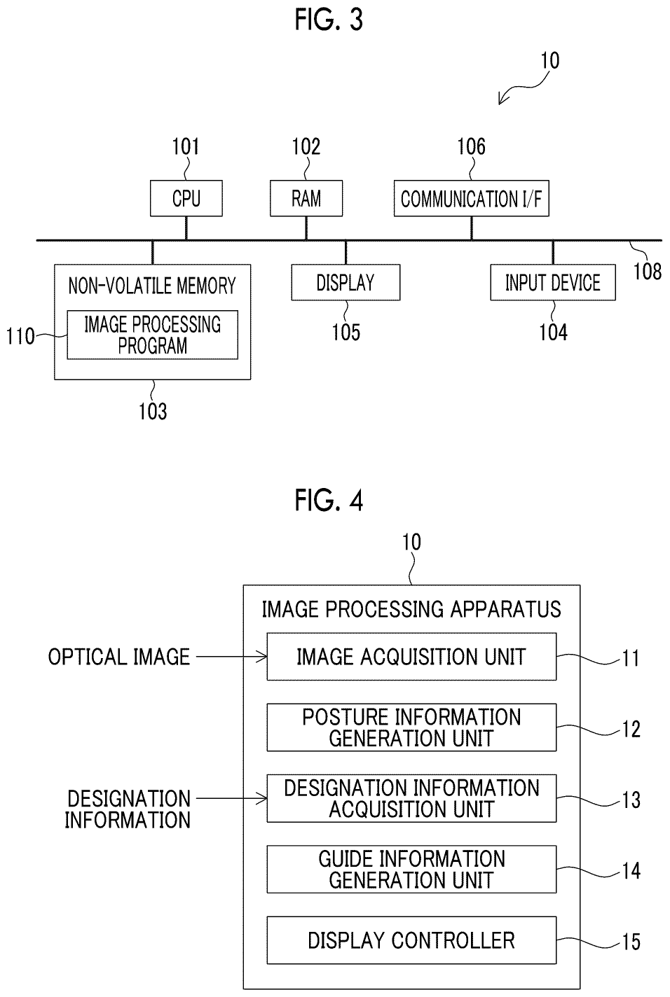

FIG. 3 is a diagram showing an example of a hardware configuration of the image processing apparatus 10. The image processing apparatus 10 includes a central processing unit (CPU) 101, a random-access memory (RAM) 102, a non-volatile memory 103, an input device 104 including a keyboard and a mouse, a display 105, and a communication interface 106. These pieces of hardware are connected to a bus 108.

The display 105 may be a touch panel display. The communication interface 106 is an interface for the image processing apparatus 10 to communicate with the radiography apparatus 20 and the optical camera 30. The communication method may be either wired or wireless. For the wireless communication, it is possible to apply a method conforming to existing wireless communication standards such as, for example, Wi-Fi (registered trademark) and Bluetooth (registered trademark).

The non-volatile memory 103 is a non-volatile storage medium such as a hard disk or a flash memory. The non-volatile memory 103 stores an image processing program 110. The RAM 102 is a work memory for the CPU 101 to execute processing. The CPU 101 loads the image processing program 110 stored in the non-volatile memory 103 into the RAM 102, and executes processing in accordance with the image processing program 110. The CPU 101 is an example of a “processor” in the disclosed technology.

FIG. 4 is a functional block diagram showing an example of a functional configuration of the image processing apparatus 10. The image processing apparatus 10 includes an image acquisition unit 11, a posture information generation unit 12, a designation information acquisition unit 13, a guide information generation unit 14, and a display controller 15. In a case in which the CPU 101 executes the image processing program 110, the image processing apparatus 10 functions as the image acquisition unit 11, the posture information generation unit 12, the designation information acquisition unit 13, the guide information generation unit 14, and the display controller 15.

The image acquisition unit 11 acquires an optical image captured by the optical camera 30. The optical image is a series of still images or videos obtained by continuously imaging a positioning process of an examinee in a videofluoroscopic examination of swallowing.

The posture information generation unit 12 extracts a plurality of feature points of the body of the examinee based on the optical image acquired by the image acquisition unit 11. The posture information generation unit 12 extracts characteristic parts of the body, such as the eyes, the cars, the nose, the shoulder joints, and the greater trochanters of the examinee, as the feature points using known face recognition technology and posture estimation technology. The posture information generation unit 12 generates a line segment determined based on any of a plurality of feature points extracted from the optical image as posture information.

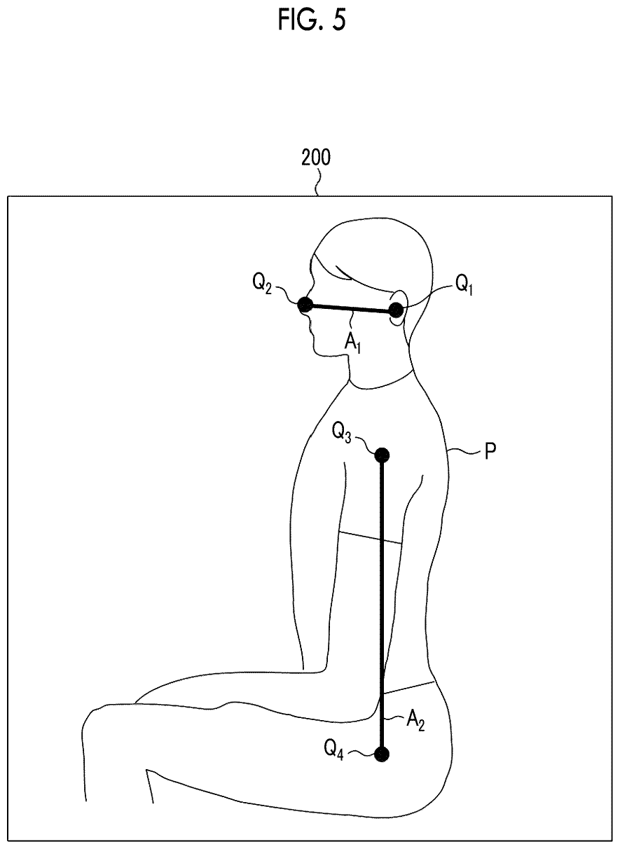

FIG. 5 is a diagram showing an example of posture information generation processing by the posture information generation unit 12. The posture information generation unit 12 extracts the eyes, the cars, the nose, the mouth, the joints, and other characteristic parts of the examinee P captured in the optical image 200 and geometrically determined points from the characteristic parts as the feature points. For example, the posture information generation unit 12 extracts a feature point Q₁ corresponding to the ears and a feature point Q₂ corresponding to the nose. The posture information generation unit 12 generates a line segment A₁ connecting the feature points Q₁ and Q₂ as first posture information. The line segment A₁ as the first posture information indicates the orientation of the face of the examinee P.

Also, for example, the posture information generation unit 12 extracts the midpoint of the left and right shoulder joints as a feature point Q₃ and extracts the midpoint of the left and right greater trochanters as a feature point Q₄. The posture information generation unit 12 generates a line segment A₂ connecting the feature points Q₃ and Q₄ as second posture information. The line segment A₂ as the second posture information indicates the orientation of the spine of the examinee P. In the optical image 200 shown in FIG. 5, the right shoulder joint and the right greater trochanter of the examinee P present on the deeper side in the depth direction are hidden and not captured in the image, but the posture information generation unit 12 is capable of extracting feature points even for parts that are not captured due to being hidden in the optical image.

The optical image 200 is a real-time image of the examinee P, and the processing by the posture information generation unit 12 is real-time processing. That is, the posture information generated by the posture information generation unit 12 indicates the current posture of the examinee P. It should be noted that the first posture information and the second posture information are merely examples, and it is also possible to use a line segment determined by the feature points other than the feature points Q₁ to Q₄ as the posture information.

In a case in which the position of the feature point is moved in accordance with the posture change of the examinee P, the posture information generation unit 12 extracts the feature point after the movement and generates a line segment determined from the feature point after the movement as new posture information. That is, the posture information generation unit 12 causes the posture information to follow the posture change of the examinee P.

The designation information acquisition unit 13 acquires designation information for designating the posture to be taken by the examinee in a videofluoroscopic examination of swallowing. The designation information can also be said to be information for designating the orientation (angle) of the line segment as guide information generated by the guide information generation unit 14. Details of the guide information will be described later.

An imaging menu for a radiation image to be captured for the examinee in a videofluoroscopic examination of swallowing includes designation information for designating a posture (for example, a face orientation (angle), a spine orientation (angle), and the like) to be taken by the examinee. The designation information acquisition unit 13 may acquire designation information included in an imaging menu.

In addition, the designation information can be input to the image processing apparatus 10 by voice or an operation of the input device 104 by an imaging person who captures a radiation image, such as a doctor or a radiologist. The input of the designation information by voice is performed through a microphone. The designation information acquisition unit 13 may acquire designation information input by voice or an operation of the input device 104.

The guide information generation unit 14 generates guide information for guiding a posture to be taken by the examinee in a videofluoroscopic examination of swallowing. The guide information generation unit 14 generates, as guide information, a line segment corresponding to the line segment as the posture information. A line segment corresponding to the line segment as the posture information may be, for example, a line segment having a point of contact with the line segment as the posture information.

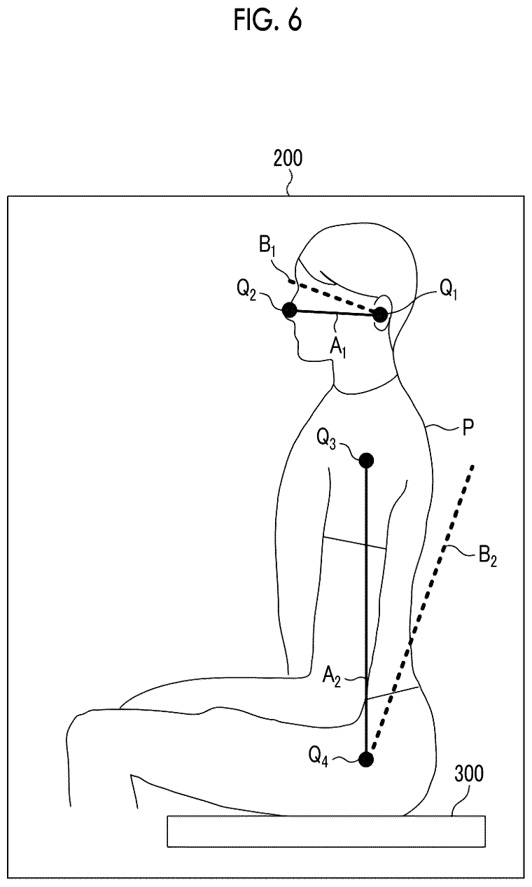

FIG. 6 is a diagram showing an example of guide information generation processing by the guide information generation unit 14. The guide information generation unit 14 generates a line segment B₁ having the feature point Q₁ closest to a support portion that supports the body of the examinee, as a starting point, among the feature points Q₁ and Q₂ present at both ends of the line segment A₁ as the first posture information indicating the current orientation of the face of the examinee P, as first guide information for guiding the orientation of the face of the examinee P. As illustrated in FIG. 6, in a case in which the examinee P sits on a seat surface 300, the seat surface 300 is a “support portion that supports the body of the examinee”. The guide information generation unit 14 sets the orientation (angle) of the line segment B₁ as the first guide information based on the designation information acquired by the designation information acquisition unit 13. The angle of the line segment as the guide information may be, for example, based on a reference of the horizontal direction (0°). For example, in a case in which the designation information designates that the orientation of the face of the examinee should be 30°, the guide information generation unit 14 generates, as the first guide information, the line segment B₁ that is inclined by 30° with respect to the horizontal direction and has the feature point Q₁ as a starting point.

The guide information generation unit 14 generates a line segment B₂ having the feature point Q₄ closest to a support portion that supports the body of the examinee, as a starting point, among the feature points Q₃ and Q₄ present at both ends of the line segment A₂ as the second posture information indicating the current orientation of the spine of the examinee P, as second guide information for guiding the orientation of the spine of the examinee P. The guide information generation unit 14 sets the orientation (angle) of the line segment B₂ as the second guide information based on the designation information acquired by the designation information acquisition unit 13. For example, in a case in which the designation information designates that the orientation of the spine of the examinee should be 120°, the guide information generation unit 14 generates, as the second guide information, the line segment B₂ that is inclined by 120° with respect to the horizontal direction and has the feature point Q₄ as a starting point. It should be noted that the first guide information and the second guide information described above are merely examples, and the guide information generation unit 14 generates guide information according to the posture information.

In a case in which the position of the feature point is moved in accordance with the posture change of the examinee P, the guide information generation unit 14 generates a line segment having the feature point after the movement as a starting point as new guide information. That is, the guide information generation unit 14 causes the guide information to follow the posture change of the examinee P.

The display controller 15 performs control such that the posture information generated by the posture information generation unit 12 and the guide information generated by the guide information generation unit 14 are displayed on the display 105. For example, as shown in FIG. 6, the display controller 15 may display the line segments A₁ and A₂ as the posture information and the line segments B₁ and B₂ as the guide information to be superimposed on the optical image of the examinee P. The line segment as the posture information and the line segment as the guide information may be displayed in different modes. For example, the line segment as the posture information and the line segment as the guide information may differ from each other in at least one of a line type, a color, or a thickness. Accordingly, it is possible to easily identify whether each line segment indicates posture information or guide information.

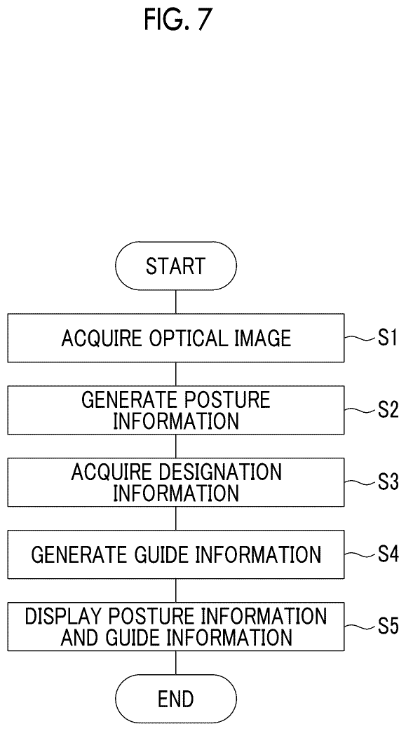

FIG. 7 is a flowchart showing an example of a flow of processing performed by the CPU 101 executing the image processing program 110.

In Step S1, the image acquisition unit 11 acquires an optical image captured by the optical camera 30. The optical image is a series of still images or videos obtained by continuously imaging a positioning process of an examinee in a videofluoroscopic examination of swallowing.

In Step S2, the posture information generation unit 12 extracts a plurality of feature points of the body of the examinee based on the optical image acquired in Step S1, and generates a line segment determined based on any of the plurality of extracted feature points as posture information.

In Step S3, the designation information acquisition unit 13 acquires designation information for designating the posture to be taken by the examinee in a videofluoroscopic examination of swallowing. The designation information can also be said to be information for designating the orientation (angle) of the line segment as guide information generated by the guide information generation unit 14.

In Step S4, the guide information generation unit 14 generates guide information for guiding a posture to be taken by the examinee in a videofluoroscopic examination of swallowing. The guide information generation unit 14 generates, as guide information, a line segment corresponding to the line segment as the posture information. The guide information generation unit 14 sets the orientation (angle) of the line segment as guide information based on the designation information acquired in Step S3.

In Step S5, the display controller 15 performs control such that the posture information generated in Step S2 and the guide information generated in Step S4 are displayed on the display 105. For example, as shown in FIG. 6, the display controller 15 may display the line segments A₁ and A₂ as the posture information and the line segments B₁ and B₂ as the guide information to be superimposed on the optical image of the examinee P.

As described above, the image processing apparatus 10 according to the embodiment of the disclosed technology acquires an optical image of an examinee, extracts a plurality of feature points of the body of the examinee based on the optical image, generates a line segment determined based on any of the plurality of feature points as posture information, which is information indicating a posture of the examinee, generates a line segment corresponding to the line segment as the posture information as guide information, which is information for guiding a posture to be taken by the examinee, and displays the posture information and the guide information.

The videofluoroscopic examination of swallowing is performed by reproducing a usual eating posture of the examinee. For example, in a case in which the examinee is in a bedridden state, the examination is performed with the examinee in a lying posture. In addition, since examinees with swallowing disorders have a habit of eating with their face tilted slightly upwards, an examination is conducted to determine whether or not aspiration occurs in a case in which the examinee tilts their face slightly upwards. In a case of capturing a radiation image, the positioning of the examinee is performed to reproduce a usual eating posture of the examinee. Currently, it is checked whether or not the posture of the examinee is set to a posture as designated by the examinee through the radiation image. That is, the examinee is irradiated with radiation for positioning purposes.

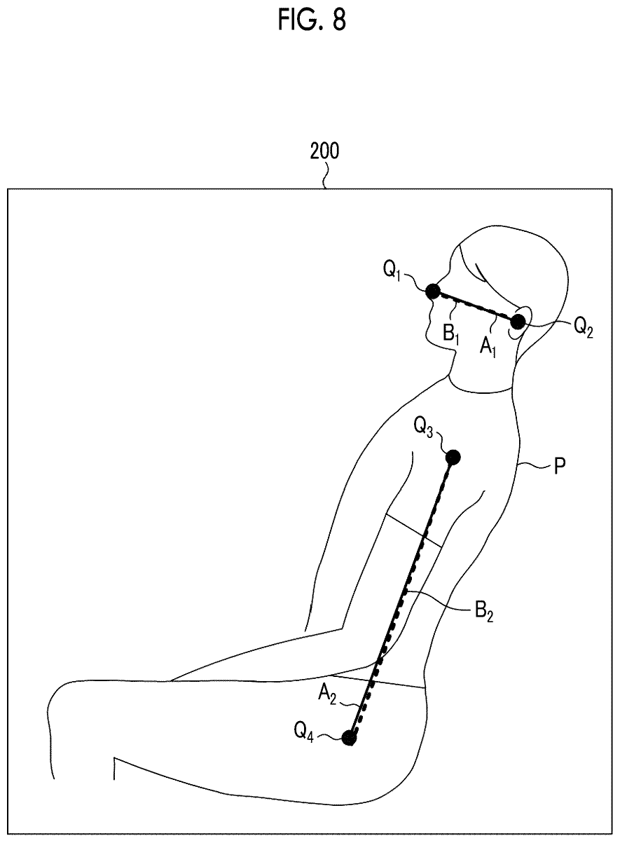

With the image processing apparatus 10 according to the present embodiment, since the information for guiding the posture to be taken by the examinee is displayed together with the posture information indicating the current posture of the examinee, it is possible to support the positioning of the examinee without performing irradiation with radiation. For example, as shown in FIG. 6, while the posture information and the guide information are displayed, the positioning of the examinee is performed such that the posture of the examinee is the posture indicated by the guide information. FIG. 8 is a diagram showing posture information and guide information in a state in which positioning is completed. With the image processing apparatus 10 according to the present embodiment, it becomes easy to perform positioning to accurately match a designated posture. Accordingly, it is possible to secure the accuracy and reproducibility of the examination.

Further, the image processing apparatus 10 displays the posture information and the guide information to be superimposed on the optical image. Accordingly, it is possible to more clearly show the current posture of the examinee and the posture to be taken by the examinee. Furthermore, the image processing apparatus 10 causes the posture information and the guide information to follow a posture change of the examinee. Accordingly, it is possible to more effectively support the positioning of the examinee.

Further, the image processing apparatus 10 generates, as the line segment as the guide information, a line segment having a feature point closest to a support portion that supports the body of the examinee as a starting point, among feature points present at both ends of the line segment as the posture information. When the posture of the examinee changes, the examinee often rotates about an axis of a feature point close to the support portion that supports the body of the examinee. Therefore, among the feature points present at both ends of a line segment as posture information, the feature point closer to the support portion that supports the body of the examinee often has a smaller movement amount when the posture of the examinee changes. By using a line segment having a feature point close to the support portion that supports the body of the examinee as a starting point as the line segment as the guide information, it is possible to provide the guide information that is suitable for the characteristics of the movement of the examinee.

### Second Embodiment

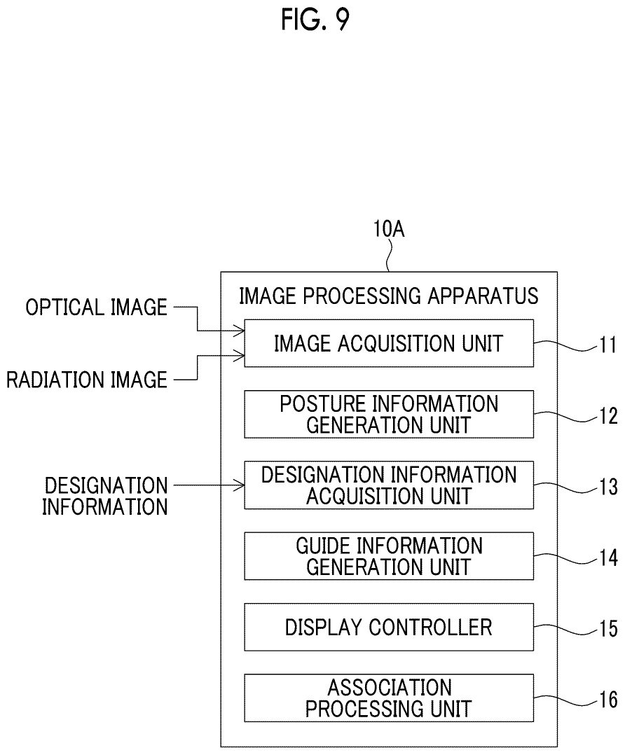

FIG. 9 is a functional block diagram showing an example of a functional configuration of an image processing apparatus 10A according to a second embodiment of the disclosed technology. The image processing apparatus 10A differs from the image processing apparatus 10 (see FIG. 4) according to the first embodiment in that the image processing apparatus 10A further includes an association processing unit 16.

In the image processing apparatus 10A according to the second embodiment, the image acquisition unit 11 acquires not only optical images but also a series of radiation images that are continuously captured with guidance from guide information. A series of radiation images are captured by performing continuous irradiation with radiation in the radiography apparatus 20. A series of radiation images are obtained by fluoroscopically imaging the process in which an examinee chews and swallows a sample in a videofluoroscopic examination of swallowing. In a case of capturing a radiation image, the examinee is positioned based on the guide information.

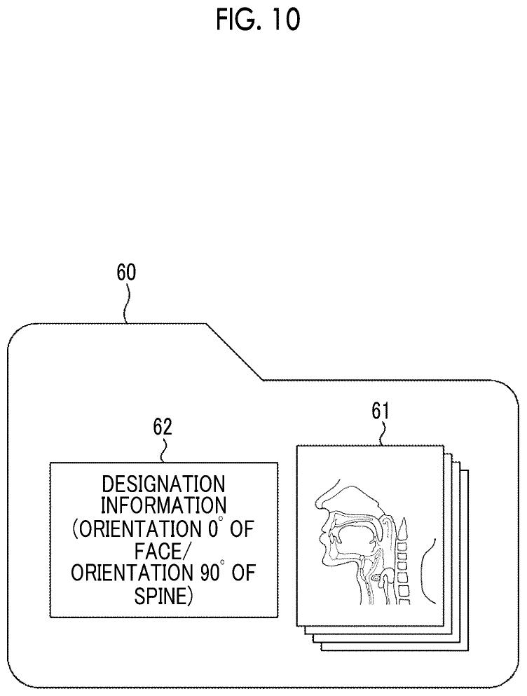

The association processing unit 16 associates the designation information acquired by the designation information acquisition unit 13 with a series of radiation images. The designation information is information for designating a posture to be taken by the examinee. For example, the orientation of the face of the examinee or the orientation of the spine of the examinee at the time of capturing the radiation image is designated by the designation information. A series of radiation images captured with guidance from the guide information are obtained by fluoroscopically imaging the examinee in a posture designated by the designation information. For example, the association processing unit 16 may associate a series of radiation images with the designation information by generating a data file 60 including designation information 62 and image group data 61 including a series of radiation images, as shown in FIG. 10.

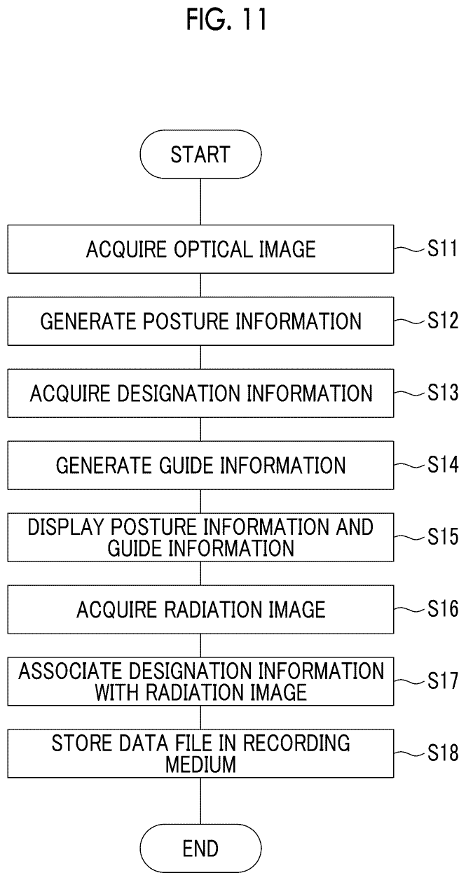

FIG. 11 is a flowchart showing an example of a flow of processing performed by the CPU 101 executing the image processing program 110 in the image processing apparatus 10A according to the second embodiment. Note that each of the processes from Step S11 to Step S15 is the same as each of the processes from Step S1 to Step S5 in the flowchart (see FIG. 7) according to the first embodiment described above, and therefore a description of these processes will be omitted.

In Step S16, the image acquisition unit 11 acquires a series of radiation images that are continuously captured with guidance from guide information. A series of radiation images are obtained by fluoroscopically imaging the examinee in a posture designated by the designation information acquired in Step S13.

In Step S17, the association processing unit 16 associates the designation information acquired in Step S13 with the series of radiation images acquired in Step S16.

In Step S18, the association processing unit 16 stores a data file in which the designation information and the series of radiation images are associated with each other in a recording medium. The recording medium in which the data file is stored may be the non-volatile memory 103 included in the image processing apparatus 10A, or may be a recording medium included in an external image server (not shown).

As described above, the image processing apparatus 10A according to the second embodiment of the disclosed technology sets the orientation of a line segment as guide information based on designation information for designating the posture to be taken by the examinee, acquires a series of radiation images continuously captured with guidance from the guide information for the examinee, and associates the designation information with the series of radiation images.

In a videofluoroscopic examination of swallowing, radiation images may be captured for each case in which the posture of the examinee is changed, and a data file of the radiation images may be generated for each posture. In this case, since a large number of data files will be generated, it is desirable to be able to ascertain which data file corresponds to which posture. It is troublesome to manually associate radiation images with postures, and there is a likelihood that the association is incorrectly performed. With the image processing apparatus 10A according to the present embodiment, since the association processing unit 16 associates the designation information with the series of radiation images, it is possible to perform accurate association between the radiation image and the posture without placing a burden on the user. In the above description, a case in which one piece of designation information is associated with a series of radiation images has been described as an example, but in a case in which radiation images are continuously captured while sequentially changing the posture of the examinee, a plurality of pieces of designation information may be associated with the series of radiation images. That is, certain designation information is associated with some of frames of a video composed of a series of radiation images, and different designation information is associated with some of the other frames. In the following, a case in which the image processing apparatus 10A associates a plurality of pieces of designation information with a series of radiation images will be described.

The designation information acquisition unit 13 sequentially acquires a plurality of pieces of designation information in which each of postures to be taken by the examinee during an imaging period of a series of radiation images is designated. The plurality of pieces of designation information can also be said to be information for designating each of the orientations (angles) of the line segments as guide information generated by the guide information generation unit 14.

The guide information generation unit 14 sets the orientation (angle) of each of the line segments as each of the plurality of pieces of guide information based on the plurality of pieces of designation information.

The display controller 15 performs control such that the plurality of pieces of guide information are sequentially displayed on the display 105 together with the posture information.

The association processing unit 16 performs, for each piece of the plurality of pieces of designation information, processing of associating one piece of the plurality of pieces of designation information with some radiation images out of a series of radiation images, the some radiation images being captured with guidance from guide information generated based on the one piece of designation information. The association processing unit 16 stores a data file in which the plurality of pieces of designation information and the series of radiation images are associated with each other in a recording medium.

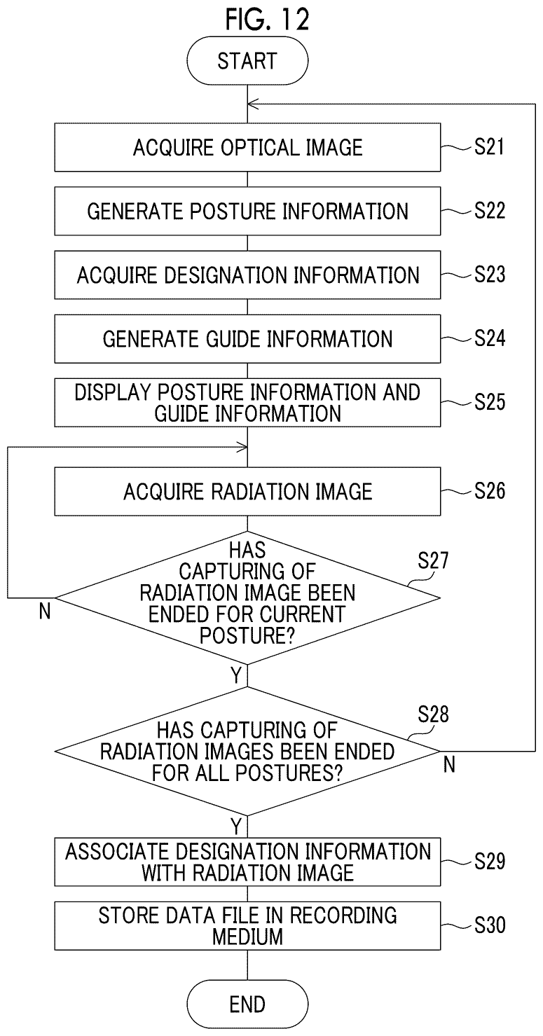

FIG. 12 is a flowchart showing an example of a flow of processing performed by the CPU 101 executing the image processing program 110 in the image processing apparatus 10A. Each of the processes from Step S21 to Step S25 is the same as each of the processes from Step S1 to Step S5 in the flowchart (see FIG. 7) according to the first embodiment described above, and therefore a description of these processes will be omitted.

Note that, in Step S23, one piece of the plurality of pieces of designation information is acquired. In Step S24, one piece of guide information is generated based on the one piece of designation information acquired in Step S23. In Step S25, the one piece of guide information generated in Step S24 is displayed on the display 105 together with the posture information.

In Step S26, the image acquisition unit 11 acquires a series of radiation images continuously captured with guidance from the guide information generated in Step S24. A series of radiation images are obtained by fluoroscopically imaging the examinee in a posture designated by the designation information acquired in Step S23.

In Step S27, the CPU 101 determines whether or not the capturing of the radiation image has been ended for the current posture. An imaging person who captures a radiation image, such as a doctor or a radiologist, can input status information indicating whether or not the capturing of the radiation image has been ended for the current posture, for example, by operating the input device 104. The CPU 101 may determine whether or not the capturing of the radiation image has been ended for the current posture based on the status information. In a case in which it is determined that the capturing of the radiation image has been ended for the current posture, the process proceeds to Step S28, and in a case in which it is determined that the capturing of the radiation image has not been ended for the current posture, the process returns to Step S26.

In Step S28, the CPU 101 determines whether or not the capturing of the radiation images has been ended for all postures. An imaging person who captures a radiation image, such as a doctor or a radiologist, can input status information indicating whether or not the capturing of the radiation images has been ended for all postures, for example, by operating the input device 104. The CPU 101 may determine whether or not the capturing of the radiation image has been ended for all postures based on the status information. In a case in which it is determined that the capturing of the radiation image has been ended for all postures, the process proceeds to Step S29, and in a case in which it is determined that the capturing of the radiation image has not been ended for all postures, the process returns to Step S21. That is, each of the processes from Step S21 to Step S27 is repeatedly performed until the capturing of the radiation images ends for all postures.

In Step S23 executed for the second and subsequent times, new designation information different from the designation information previously acquired is acquired. In Step S24, new guide information is generated based on the new designation information. In Step S25, the new guide information is displayed on the display 105 together with the posture information. In Step S26, a series of radiation images continuously captured with guidance from the new guide information are acquired. The radiation images are captured continuously until the capturing of the radiation images ends for all postures.

In Step S29, the association processing unit 16 performs, for each piece of the plurality of pieces of designation information, processing of associating one piece of the plurality of pieces of designation information with some radiation images out of a series of radiation images, the some radiation images being captured with guidance from guide information generated based on the one piece of designation information. That is, the association processing unit 16 performs processing of associating the designation information acquired in Step S23 with the series of radiation images acquired in Step S26 for each repeating unit in repetition processing in which the processes from Step S21 to Step S27 are a repeating unit.

In Step S30, the association processing unit 16 stores a data file in which a plurality of pieces of designation information and a series of radiation images are associated with each other in a recording medium.

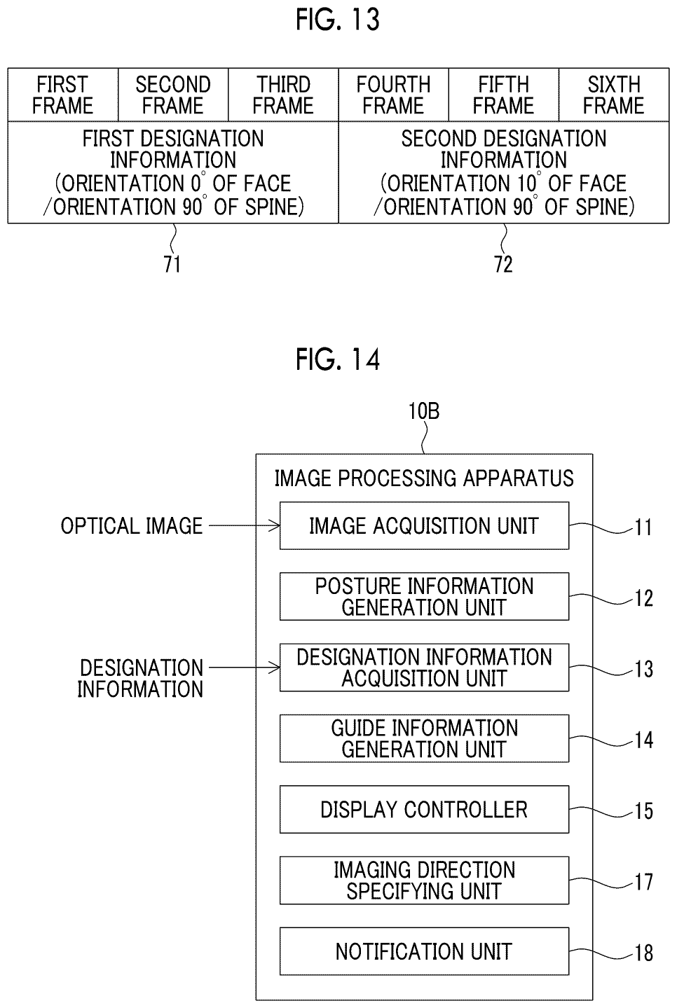

FIG. 13 is a diagram showing an example of association processing performed by the association processing unit 16. The series of radiation images acquired by the image acquisition unit 11 constitute each frame of a video (hereinafter referred to as an examination video) composed of the series of radiation images. As an example, FIG. 13 shows a case in which first designation information 71 is associated with each frame from a first frame to a third frame of the examination video, and second designation information 72 is associated with each frame from a fourth frame to a sixth frame of the examination video.

The first designation information 71 is information indicating that the orientation of the face of the examinee should be 0° and the orientation of the spine of the examinee should be 90°. The association processing unit 16 associates the first designation information 71 with each frame from the first frame to the third frame captured with guidance from the guide information generated based on the first designation information 71. The second designation information 72 is information indicating that the orientation of the face of the examinee should be 10° and the orientation of the spine of the examinee should be 90°. The association processing unit 16 associates the second designation information 72 with each frame of the fourth frame to the sixth frame captured with guidance from the guide information generated based on the second designation information 72.

As described above, the image processing apparatus 10A according to the present embodiment sets an orientation of each of line segments as each of a plurality of pieces of the guide information based on a plurality of pieces of designation information for designating postures to be taken by the examinee, respectively, acquires a series of radiation images continuously captured with guidance from each of the plurality of pieces of guide information, and performs, for each piece of the plurality of pieces of designation information, processing of associating one piece of the plurality of pieces of designation information with some radiation images out of the series of radiation images, the some radiation images being captured with guidance from the guide information generated based on the one piece of designation information.

In a videofluoroscopic examination of swallowing, radiation images may be continuously captured while the posture of the examinee is changed, and a plurality of postures may be mixed in one examination video. In this case, it is desirable to accurately associate the posture of the examinee with the frame related to that posture such that it is possible to ascertain which portion of the series of radiation images corresponds to which posture.

With the image processing apparatus 10A according to the present embodiment, since the processing of associating one piece of the plurality of pieces of designation information with some radiation images that are captured with guidance from guide information generated based on the one piece of designation information out of a series of radiation images is performed for each piece of the plurality of pieces of designation information, it is possible to accurately associate the frame with the posture without placing a burden on the user. With the image processing apparatus 10A according to the present embodiment, for example, in a case of playing back the examination video, it is possible to cue up a portion related to a desired posture.

In the above description, a case in which the designation information and the radiation image are associated with each other has been described as an example, but the disclosed technology is not limited to this aspect. Information indicating the orientation (angle) of the line segment as the posture information at the point in time at which the positioning of the examinee is completed may be associated with the radiation image instead of the designation information or together with the designation information. Information indicating the orientation (angle) of the line segment as the posture information indicates an actual posture of a subject to be examined at that time.

### Third Embodiment

FIG. 14 is a functional block diagram showing an example of a functional configuration of an image processing apparatus 10B according to a third embodiment of the disclosed technology. The image processing apparatus 10B differs from the image processing apparatus 10 (see FIG. 4) according to the first embodiment in that the image processing apparatus 10B further includes an imaging direction specifying unit 17 and a notification unit 18.

The imaging direction specifying unit 17 specifies an imaging direction of a radiation image to be captured for the examinee based on at least some of the plurality of feature points extracted by the posture information generation unit 12. The imaging direction specifying unit 17 may specify the imaging direction based on a positional relationship between feature points corresponding to the left and right eyes of the examinee, for example. It is also possible to specify the imaging direction based on a positional relationship between feature points corresponding to the left and right cars or a positional relationship between feature points corresponding to the left and right shoulder joints. The imaging direction specifying unit 17 specifies, for example, whether the imaging direction is a direction in which the examinee is imaged from the front or a direction in which the examinee is imaged from the side.

The feature points are extracted from the optical image captured by the optical camera 30. However, in a case in which a relationship between the imaging direction of the optical image by the optical camera 30 and the imaging direction of the radiation image by the radiography apparatus 20 is known, it is possible to specify the imaging direction of the radiation image based on the feature points.

In a case in which the imaging direction specified by the imaging direction specifying unit 17 is different from the designated imaging direction, the notification unit 18 issues an alert to notify that the imaging direction of the radiation image is different from the designated imaging direction. The notification unit 18 may, for example, issue an alert by displaying a predetermined message on the display 105 or may issue an alert by outputting a predetermined voice from a speaker (not shown). The designation information acquired by the designation information acquisition unit 13 includes information for designating an imaging direction of the radiation image. The notification unit 18 issues an alert in a case in which the imaging direction specified by the imaging direction specifying unit 17 is different from the imaging direction indicated by the designation information.

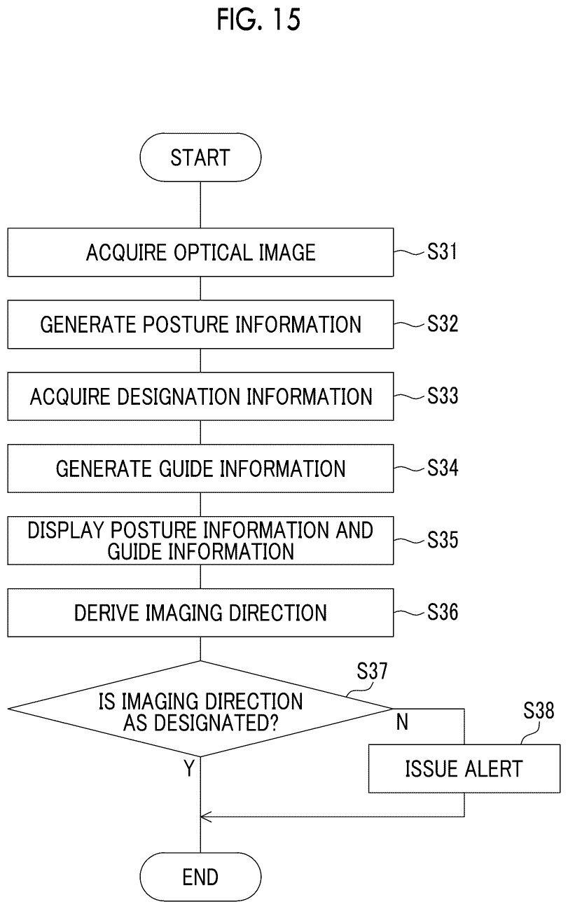

FIG. 15 is a flowchart showing an example of a flow of processing performed by the CPU 101 executing the image processing program 110 in the image processing apparatus 10B according to the third embodiment. Note that each of the processes from Step S31 to Step S35 is the same as each of the processes from Step S1 to Step S5 in the processing (see FIG. 7) according to the first embodiment described above, and therefore a description of these processes will be omitted. It is assumed that the designation information acquired in Step S33 includes information for designating the orientation (angle) of the line segment as guide information and information for designating the imaging direction of the radiation image.

In Step S36, the imaging direction specifying unit 17 specifies an imaging direction of a radiation image to be captured for the examinee based on at least some of the plurality of feature points extracted in Step S32.

In Step S37, the notification unit 18 determines whether or not the imaging direction specified in Step S36 matches the imaging direction indicated by the designation information acquired in Step S33. In a case in which it is determined that the imaging direction specified in Step S36 matches the imaging direction indicated by the designation information acquired in Step S33, the process ends. On the other hand, in a case in which it is determined that the imaging direction specified in Step S36 is different from the imaging direction indicated by the designation information acquired in Step S33, the process proceeds to Step S38.

In Step S38, the notification unit 18 issues an alert to notify that the imaging direction of the radiation image is different from the designated imaging direction.

With the image processing apparatus 10 according to the present embodiment, since the alert is issued in a case in which the imaging direction of the radiation image specified based on the feature points is different from the designated imaging direction, it is possible to prevent the need to recapture the radiation image in advance.

In the above first to third embodiments, a case in which the disclosed technology is applied to a videofluoroscopic examination of swallowing has been described as an example, but the disclosed technology is not limited to this aspect. The disclosed technology can be applied in any situation in which the posture of the examinee is designated.

As hardware for executing processes in each functional unit of the image processing apparatuses 10, 10A, and 10B, various processors as shown below can be used. The processor may be a CPU that executes software (programs) and functions as various processing units. Furthermore, the processor may be a programmable logic device (PLD) such as an FPGA whose circuit configuration is changeable. Moreover, the processor may have a circuit configuration that is specially designed to execute a specific process, such as an application-specific integrated circuit (ASIC).

Each functional unit of the image processing apparatuses 10, 10A, and 10B may be configured by one of the various processors described above, or may be configured by a combination of the same or different kinds of two or more processors (for example, a combination of a plurality of FPGAs or a combination of the CPU and the FPGA). In addition, a plurality of functional units may be configured by one processor.

In the above embodiments, the image processing program 110 has been described as being stored (installed) in the non-volatile memory 103 in advance; however, the present disclosure is not limited thereto. The image processing program 110 may be provided in a form recorded in a recording medium such as a compact disc read-only memory (CD-ROM), a digital versatile disc read-only memory (DVD-ROM), and a universal serial bus (USB) memory. In addition, the image processing program 110 may be configured to be downloaded from an external device via a network.

Regarding the first to fourth embodiments, the following supplementary notes are further disclosed.

### Supplementary Note 1

An image processing apparatus comprising at least one processor,
in which the processor is configured to:
   acquire an optical image of an examinee;
   extract a plurality of feature points of a body of the examinee based on the optical image;
   generate a line segment determined based on any of the plurality of feature points as posture information, which is information indicating a posture of the examinee;
   generate a line segment corresponding to the line segment as the posture information as guide information, which is information for guiding a posture to be taken by the examinee; and
   display the posture information and the guide information.

### Supplementary Note 2

The image processing apparatus according to Supplementary Note 1,
in which the processor is configured to display the posture information and the guide information to be superimposed on the optical image.

### Supplementary Note 3

The image processing apparatus according to Supplementary Note 1 or 2,
in which the processor is configured to cause the posture information and the guide information to follow a posture change of the examinee.

### Supplementary Note 4

The image processing apparatus according to any one of Supplementary Notes 1 to 3,
in which the processor is configured to generate, as the guide information, a line segment having a feature point closest to a support portion that supports the body of the examinee as a starting point, among feature points present at both ends of the line segment as the posture information.

### Supplementary Note 5

The image processing apparatus according to any one of Supplementary Notes 1 to 4,
in which the processor is configured to set an orientation of the line segment as the guide information based on designation information for designating a posture to be taken by the examinee.

### Supplementary Note 6

The image processing apparatus according to Supplementary Note 5,
in which the processor is configured to acquire the designation information included in an imaging menu for a radiation image to be captured for the examinee.

### Supplementary Note 7

The image processing apparatus according to Supplementary Note 5,
in which the processor is configured to acquire the designation information input by an operation of an input unit or voice.

### Supplementary Note 8

The image processing apparatus according to any one of Supplementary Notes 1 to 7,
in which the processor is configured to:
   extract an ear of the examinee as a first feature point and extract an eye of the examinee as a second feature point based on the optical image; and
   generate a line segment connecting the first feature point and the second feature point as the posture information.

### Supplementary Note 9

The image processing apparatus according to any one of Supplementary Notes 1 to 8,
in which the processor is configured to:
   extract a midpoint of left and right shoulder joints of the examinee as a third feature point and extract a midpoint of left and right greater trochanters of the examinee as a fourth feature point based on the optical image; and
   generate a line segment connecting the third feature point and the fourth feature point as the posture information.

### Supplementary Note 10

The image processing apparatus according to any one of Supplementary Notes 1 to 9,
in which the processor is configured to:
   set an orientation of the line segment as the guide information based on designation information for designating a posture to be taken by the examinee;
   acquire a series of radiation images continuously captured with guidance from the guide information for the examinee; and
   associate the designation information with the series of radiation images.

### Supplementary Note 11

The image processing apparatus according to any one of Supplementary Notes 1 to 10,
in which the processor is configured to:
   set an orientation of each of line segments as each of a plurality of pieces of the guide information based on a plurality of pieces of designation information for designating postures to be taken by the examinee, respectively;
   acquire a series of radiation images continuously captured with guidance from each of the plurality of pieces of guide information; and
   perform, for each piece of the plurality of pieces of designation information, processing of associating one piece of the plurality of pieces of designation information with some radiation images out of the series of radiation images, the some radiation images being captured with guidance from the guide information generated based on the one piece of designation information.

### Supplementary Note 12

The image processing apparatus according to any one of Supplementary Notes 1 to 11,
in which the processor is configured to:
   specify an imaging direction of a radiation image to be captured for the examinee based on at least some of the plurality of feature points; and
   issue an alert in a case in which the specified imaging direction is different from a designated direction.

What is claimed is:

1. An image processing apparatus comprising at least one processor,
wherein the processor is configured to:
acquire an optical image of an examinee;
extract a plurality of feature points of a body of the examinee based on the optical image;
generate a line segment determined based on any of the plurality of feature points as posture information, which is information indicating a posture of the examinee;
generate a line segment corresponding to the line segment as the posture information as guide information, which is information for guiding a posture to be taken by the examinee; and
display the posture information and the guide information.

2. The image processing apparatus according to claim 1,
wherein the processor is configured to display the posture information and the guide information to be superimposed on the optical image.

3. The image processing apparatus according to claim 1,
wherein the processor is configured to cause the posture information and the guide information to follow a posture change of the examinee.

4. The image processing apparatus according to claim 1,
wherein the processor is configured to generate, as the guide information, a line segment having a feature point closest to a support portion that supports the body of the examinee as a starting point, among feature points present at both ends of the line segment as the posture information.

5. The image processing apparatus according to claim 1,
wherein the processor is configured to set an orientation of the line segment as the guide information based on designation information for designating a posture to be taken by the examinee.

6. The image processing apparatus according to claim 5,
wherein the processor is configured to acquire the designation information included in an imaging menu for a radiation image to be captured for the examinee.

7. The image processing apparatus according to claim 5,
wherein the processor is configured to acquire the designation information input by an operation of an input unit or voice.

8. The image processing apparatus according to claim 1,
wherein the processor is configured to:
extract an ear of the examinee as a first feature point and extract an eye of the examinee as a second feature point based on the optical image; and
generate a line segment connecting the first feature point and the second feature point as the posture information.

9. The image processing apparatus according to claim 1,
wherein the processor is configured to:
extract a midpoint of left and right shoulder joints of the examinee as a third feature point and extract a midpoint of left and right greater trochanters of the examinee as a fourth feature point based on the optical image; and
generate a line segment connecting the third feature point and the fourth feature point as the posture information.

10. The image processing apparatus according to claim 1,
wherein the processor is configured to:
set an orientation of the line segment as the guide information based on designation information for designating a posture to be taken by the examinee;
acquire a series of radiation images continuously captured with guidance from the guide information for the examinee; and
associate the designation information with the series of radiation images.

11. The image processing apparatus according to claim 1,
wherein the processor is configured to:
set an orientation of each of line segments as each of a plurality of pieces of the guide information based on a plurality of pieces of designation information for designating postures to be taken by the examinee, respectively;
acquire a series of radiation images continuously captured with guidance from each of the plurality of pieces of guide information; and
perform, for each piece of the plurality of pieces of designation information, processing of associating one piece of the plurality of pieces of designation information with some radiation images out of the series of radiation images, the some radiation images being captured with guidance from the guide information generated based on the one piece of designation information.

12. The image processing apparatus according to claim 1,
wherein the processor is configured to:
specify an imaging direction of a radiation image to be captured for the examinee based on at least some of the plurality of feature points; and
issue an alert in a case in which the specified imaging direction is different from a designated direction.

13. An image processing method executed by at least one processor included in an image processing apparatus, the image processing method comprising:
acquiring an optical image of an examinee;
extracting a plurality of feature points of a body of the examinee based on the optical image;
generating a line segment determined based on any of the plurality of feature points as posture information, which is information indicating a posture of the examinee;
generating a line segment corresponding to the line segment as the posture information as guide information, which is information for guiding a posture to be taken by the examinee; and
displaying the posture information and the guide information.

14. A non-transitory computer-readable storage medium storing a program for causing at least one processor included in an image processing apparatus to execute:
acquiring an optical image of an examinee;
extracting a plurality of feature points of a body of the examinee based on the optical image;
generating a line segment determined based on any of the plurality of feature points as posture information, which is information indicating a posture of the examinee;
generating a line segment corresponding to the line segment as the posture information as guide information, which is information for guiding a posture to be taken by the examinee; and
displaying the posture information and the guide information.
